(19) United States
(12) Patent Application Publication
(10) Pub. No.: US 2009/0187102 A1
(43) Pub. Date: Jul. 23, 2009

(54) METHOD AND APPARATUS FOR WIDE-SCREEN MEDICAL IMAGING

(76) Inventors: Gerois Di Marco, St. Francis, WI (US); Jason Haedt, Wauwatosa, WI (US)

Correspondence Address: DEAN D. SMALL;THE SMALL PATENT LAW GROUP LLP, 225 S. MERAMEC, STE. 725T, ST. LOUIS, MO, 63105, US

(21) Appl. No.: 12/017,220

(22) Filed: Jan. 21, 2008

(51) Int. Cl. A61B 8/00; A61B 5/00

(52) U.S. Cl. 600/437; 600/407

(57) ABSTRACT

A medical imaging system comprises a detector for detecting imaging data associated with a subject. A display has first and second dimensions and a position sensor detects an orientation of the display. The orientation is associated with the first and second dimensions. A processor module determines the orientation of the display based on the position sensor and displays information on the display in a first viewing configuration when the display is in a first orientation and in a second viewing configuration when the display is in a second orientation.

### BACKGROUND OF THE INVENTION

One or more embodiments of this invention relate generally to medical imaging systems and more particularly to displaying medical images and information using the medical imaging systems.

Medical imaging systems are becoming more sophisticated, increasing one or more of the quantity and quality of imaging data. Also, the ease of use by the operator is important, potentially allowing the operator to focus more of their time on the patient and on image collection, analysis and comparison.

During an exam, the operator may want to display data in more than one format and/or configuration. Unfortunately, the operator typically has access to only one display configuration at a time. Also, the size of the display limits both the quantity and the display configuration of the data. The operator may thus need to use multiple keystrokes to display the data differently and/or to return to a previous viewing configuration. Also, the operator often inputs data related to the patient that requires multiple different pages wherein only one page is typically displayed at a time.

Therefore, a need exists for improved display and customization of medical imaging data and associated patient information.

### BRIEF DESCRIPTION OF THE INVENTION

In one embodiment, a medical imaging system comprises a detector for detecting imaging data associated with a subject. A display has first and second dimensions and a position sensor detects an orientation of the display. The orientation is associated with the first and second dimensions. A processor module determines the orientation of the display based on the position sensor and displays information on the display in a first viewing configuration when the display is in a first orientation and in a second viewing configuration when the display is in a second orientation.

In another embodiment, an ultrasound imaging system comprises a transducer for detecting ultrasound imaging data associated with a subject. A wide-screen display has first and second dimensions that are different with respect to each other. A position sensor detects an orientation of the wide-screen display and a processor module determines the orientation of the display based on the position sensor. The processor module displays information on the wide-screen display in a first viewing configuration when the wide-screen display is in a first orientation and in a second viewing configuration when the wide-screen display is in a second orientation.

In yet another embodiment, a method for changing a viewing configuration based on a position of a display associated with a medical imaging system comprises detecting medical imaging data associated with a subject. A first position of a display is identified and is associated with one of a landscape orientation and a portrait orientation. Information is displayed on the display based on the first position and a first viewing configuration. A second position of the display is identified that is rotationally different with respect to the first position. The information is displayed on the display based on the second position and a second viewing configuration that is different than the first viewing configuration.

### BRIEF DESCRIPTION OF THE DRAWINGS

FIG. 1 is a block diagram of an ultrasound system formed in accordance with an embodiment of the present invention.

FIG. 2 illustrates a console-based ultrasound system that has a wide-screen display formed in accordance with an embodiment of the present invention.

FIG. 3 illustrates a viewing configuration of the wide-screen display in a landscape orientation in accordance with an embodiment of the present invention.

FIG. 4 illustrates another viewing configuration of the wide-screen display in a landscape orientation in accordance with an embodiment of the present invention.

FIG. 5 illustrates a viewing configuration of the wide-screen display in a portrait orientation in accordance with an embodiment of the present invention.

FIG. 6 illustrates another viewing configuration of the wide-screen display in a portrait orientation in accordance with an embodiment of the present invention.

FIG. 7 illustrates yet another viewing configuration of the wide-screen display in a portrait orientation in accordance with an embodiment of the present invention.

FIG. 8 illustrates a viewing configuration of the wide-screen display in landscape orientation that displays multiple pages of text simultaneously in accordance with an embodiment of the present invention.

FIG. 9 illustrates a viewing configuration of the wide-screen display in portrait orientation that displays pages of text in a single list format in accordance with an embodiment of the present invention.

FIG. 10 illustrates a viewing configuration wherein the wide-screen display displays a page of text and one or more images simultaneously in accordance with an embodiment of the present invention.

FIG. 11 illustrates a viewing configuration wherein the wide-screen display separately displays annotations associated with an image in accordance with an embodiment of the present invention.

FIG. 12 illustrates a miniaturized ultrasound system formed in accordance with an embodiment of the present invention.

FIG. 13 illustrates a hand carried or pocket-sized ultrasound imaging system formed in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

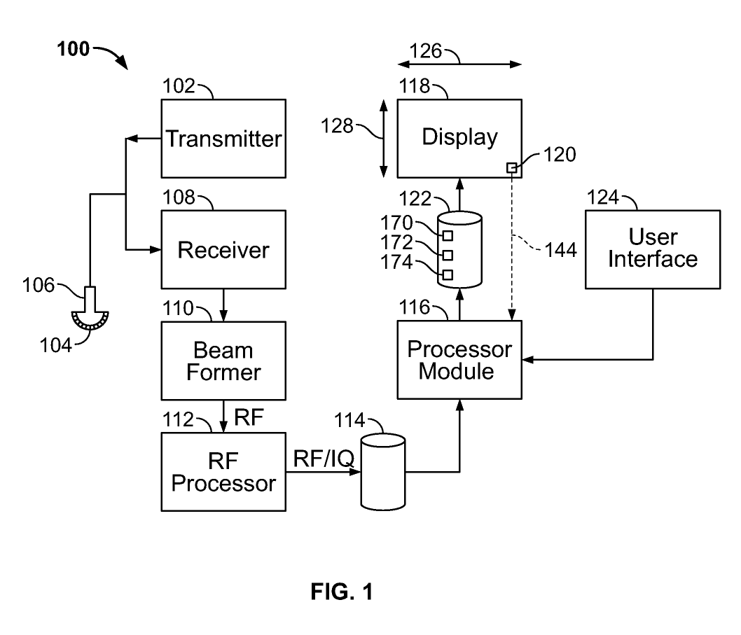

FIG. 1 illustrates an ultrasound system 100 including a transmitter 102 that drives an array of elements 104 (e.g., piezoelectric elements) within a transducer 106 to emit pulsed ultrasonic signals into a body. The elements 104 may be arranged, for example, in one or two dimensions. A variety of geometries may be used. The ultrasonic signals are back-scattered from structures in the body, like fatty tissue or muscular tissue, to produce echoes that return to the elements 104. The echoes are received by a receiver 108. The received echoes are passed through a beamformer 110 that performs beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to a memory 114 for storage.

The ultrasound system 100 also includes a processor module 116 to process the acquired ultrasound information (e.g., RF signal data or IQ data pairs) and prepare frames of ultrasound information for display on display 118. The processor module 116 is adapted to perform one or more processing operations according to a plurality of selectable ultrasound modalities on the acquired ultrasound information. Acquired ultrasound information may be processed and displayed in real-time during a scanning session as the echo signals are received. Additionally or alternatively, the ultrasound information may be stored temporarily in memory 114 or memory 122 during a scanning session and then processed and displayed in an off-line operation.

A user interface 124 may be used to input data into the system 100 and to adjust settings and control operation of the processor module 116. One or both of memory 114 and memory 122 may store two-dimensional (2D) and/or three-dimensional (3D) datasets of the ultrasound data, where such datasets are accessed to present 2D and/or 3D images. Multiple consecutive 3D datasets may also be acquired and stored over time, such as to provide real-time 3D or four-dimensional (4D) display. The images may be modified and the display settings of the display 118 also manually adjusted using the user interface 124.

The display 118 includes at least one wide-screen display for presenting patient information to the operator. The display 118 has first and second dimensions 126 and 128 that are different with respect to each other. The display 118 is configured to display one or more images, patient information, and/or user interfaces in display configurations that do not limit the resolution and/or size of the images(s) and that maximizes the amount of data (e.g. images and/or text) that may be displayed. For example, the display 118 may be a wide-screen display wherein at least one of the first and second dimensions 126 and 128 is greater than 15 inches. The display 118 may have an aspect ratio that is greater than the typical 4:5 aspect ratio of a standard, substantially square display. In the orientation illustrated in FIG. 1, also referred to as landscape orientation, the first and second dimensions 126 and 128 correspond to X and Y dimensions, respectively, of the display 118 and the first dimension 126 is longer or greater than the second dimension 128. When the display 118 is rotated 90 degrees, such that the first and second dimensions 126 and 128 correspond to Y and X dimensions, respectively, the orientation may be referred to as portrait orientation.

A position sensor 120 may be mounted within or proximate to the display 118. The position sensor 120 may be an electromechanical device or other device that senses, detects and/or indicates the orientation of the display 118 and transmits and/or provides an indication of the orientation to the processor module 116 via link 144 that may be hardwired or wireless. The position sensor 120 may alternatively be a mechanical switch that has open and closed states. The processor module 116 may thus sense the open or closed state of the position sensor 120 to identify the orientation of the display 118.

For example, the position sensor 120 may be configured to identify the current orientation of the display 118 with respect to the operator's viewpoint. The position sensor 120 senses, detects and/or indicates whether the first and second dimensions 126 and 128 correspond to X and Y dimensions, respectively, as shown, or correspond to the Y and X dimensions, respectively, associated with a relative rotation of 90 degrees. For example, the operator may rotate the display 118 to change the viewing configuration of the image data and/or other information currently being displayed to a different viewing configuration.

The operator may store one or more viewing configurations in the memory 122. For example, the operator may define one or more default viewing configurations 170 upon which the display of data in the portrait and landscape orientations is based. Alternatively, the default viewing configurations 170 may be preset and may be subsequently modified by the operator. The operator may also define, for example, first and second viewing configurations 172 and 174 that may be associated with landscape and portrait orientations of the display 118 when a particular viewing and/or acquisition protocol (not shown) is used. A protocol may thus be used to acquire and/or display particular anatomy, such as cardiac, kidney or liver, in predetermined and desired formats or representations. It should be understood that many different viewing configurations may be defined, and that different operators may customize their viewing preferences by defining personal viewing configurations.

Although the wide-screen display 118 and the position sensor 120 are discussed herein with respect to ultrasound imaging systems, it should be understood that other types of medical imaging systems may be used. For example, the wide-screen display 118 and position sensor 120 may be used with X-ray technology, computed tomography (CT), MRI, Nuclear Medicine and/or other systems that may be used for diagnostic and/or interventional procedures.

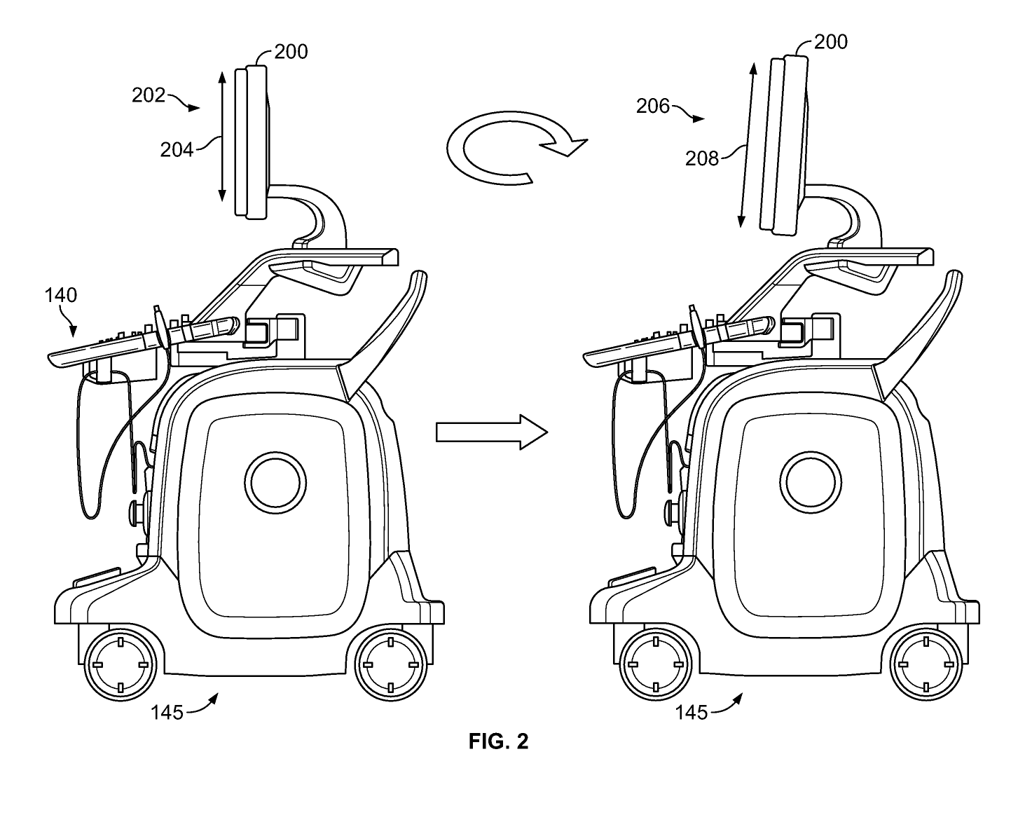

FIG. 2 illustrates a console-based ultrasound system 145 that has a wide-screen display 200. The system 145 also has a user interface 140 that includes control buttons (not shown) that may be used to control the system 145 as desired or needed, and/or as typically provided. The user interface 140 provides multiple interface options such as a keyboard and track ball that the operator may physically manipulate to interact with ultrasound data and other data that may be displayed, input information and set and change scanning parameters as well as viewing configurations. The interface options may be used for specific inputs, programmable inputs, contextual inputs, and the like.

The wide-screen display 200 has the first and second dimensions 126 and 128 as discussed in FIG. 1 wherein one of the dimensions is greater than the other. In a first position 202, the wide-screen display 200 is positioned such that a smaller or shorter dimension 204 is oriented vertically (landscape orientation). The wide-screen display 200 may be rotated 90 degrees to a second position 206 such that a greater or longer dimension 208 is oriented vertically (portrait orientation). The landscape and portrait orientations are with respect to the operator's view of the wide-screen display 200.

The position sensor 120 (as shown in FIG. 1) may automatically sense the orientation of the wide-screen display 200 and change or update the current viewing configuration accordingly. In another embodiment, the automatic sensing may be disabled such that the operator may manually control, such as from the user interface 140, when and if the data on the wide-screen display 200 is changed from one viewing configuration to another.

Because the wide-screen display 200 is larger than, for example, typical displays, information such as patient images may be displayed in a larger format. Also, if the operator is entering data, the wide-screen display 200 may use a relatively larger font for ease of viewing and/or display more entry fields at a time. For example, a typical display may display one screen of data to the operator at a time, while the wide-screen display 200 may display twice the data or multiple pages, such as two screens of data in an open-book format. This improves the workflow for the operator as the operator does not need to manually switch from one screen to the next and/or more information may be input and/or reviewed at one time. In another embodiment, the wide-screen display 200 may display images and/or data input screens while displaying touch-enabled buttons in one or more areas, such as along one vertical side or along the bottom, based on the current orientation of the wide-screen display 200. The operator may then simply rotate the wide-screen display 200 to change the way the information is displayed, such as changing the content, arrangement, relation of certain data to one another on the wide-screen display 200, and the like.

In another embodiment, the size of the wide-screen display 200 may be such that the longer dimension 208 is greater than a width of the ultrasound system 145 and/or greater than the width of an opening such as a doorway. Therefore, the ability to rotate the wide-screen display 200 may be helpful when moving the ultrasound system 145 from one location to another.

As previously discussed, the operator may customize the wide-screen display 200 based on the application being used. In one application, the operator may select a certain size of image(s) and/or define the positions of an image, multiple images, and/or images and data, on the display area of the wide-screen display as well as with respect to each other. For example, in one application the operator may define first and second screens of ultrasound information to be displayed side-by-side on the wide-screen display 200 when in landscape orientation. If the wide-screen display 200 is rotated to be in portrait orientation, the operator may define that the first and second screens of ultrasound data are displayed one above the other automatically with room along one side and/or the bottom of the wide-screen display 200 for either touchpad or data entry.

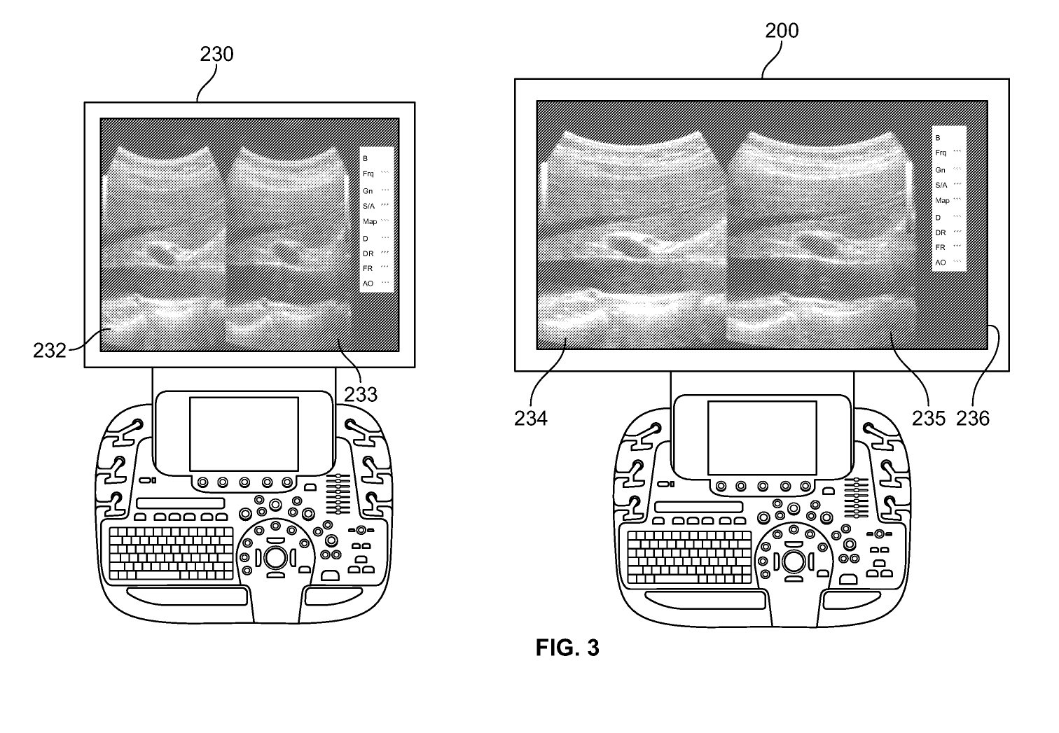

FIG. 3 illustrates a comparison of how patient data may be displayed on a standard display 230 and the wide-screen display 200 in the landscape orientation. The standard display 230 displays first and second images 232 and 233 side-by-side and the wide-screen display 200 displays first and second images 234 and 235 side-by-side. Although the diagnostic content of the images may be the same, because the wide-screen display 200 is much larger in the landscape format than the standard display 230, the first and second images 234 and 235 may be displayed in a larger size and/or with greater resolution than the first and second images 232 and 233 and thus provide an improved viewing experience for the operator. Also, more data display space 236 is available on the wide-screen display 200 for displaying metrics associated with the first and second images 234 and 235, patient information and the like, and the information may be displayed in a larger format or font. In another embodiment, the data display space 236 on the wide-screen display 200 may be used to provide touch or mouse-selectable button(s) for the operator.

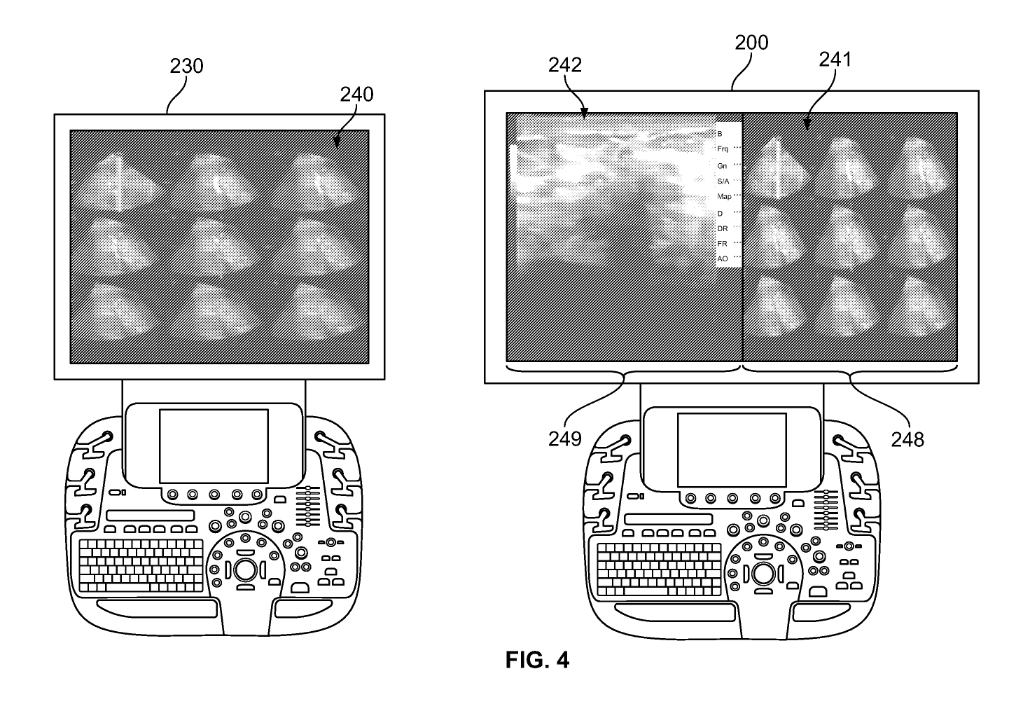

FIG. 4 illustrates another example of how the wide-screen display 200 may be configured to display more patient data than the standard display 230 in the landscape orientation. On the standard display 230, a display of nine images 240 encompasses the entire display area. The nine images 240 may be an example of tomographic ultrasound imaging (TUI) wherein ultrasound images are displayed as slices similar to images acquired using CT. Turning to the wide-screen display 200, the display of nine images 241 is displayed in one-half of the display area or in first display area 248 while an additional image 242 is displayed simultaneously in the other half of the display area or in second display area 249. Therefore, additional patient diagnostic information may be displayed allowing the operator to review more data at the same time. By way of example only, the additional image 242 may be used to display depth information within the patient (or an enlarged view of an image selected in the first display area 248). In another example, more images, such as eighteen images, may be displayed simultaneously on the wide-screen display 200.

In another embodiment, the review and/or collection of patient data may be enhanced by displaying images from more than one imaging modality simultaneously on the wide-screen display 200. For example, the wide-screen display 200 may display a live or stored ultrasound image on a portion of the display area while displaying image(s), such as of the same anatomy of the patient that were acquired using a different modality, such as CT, MRI or X-ray. For example, the image 242 may be a current ultrasound image while the nine images 241 may be CT images. Images from other modalities or imaging systems may be accessed, such as via a network or the internet from a database, manually from a portable storage medium, and/or directly from another imaging system.

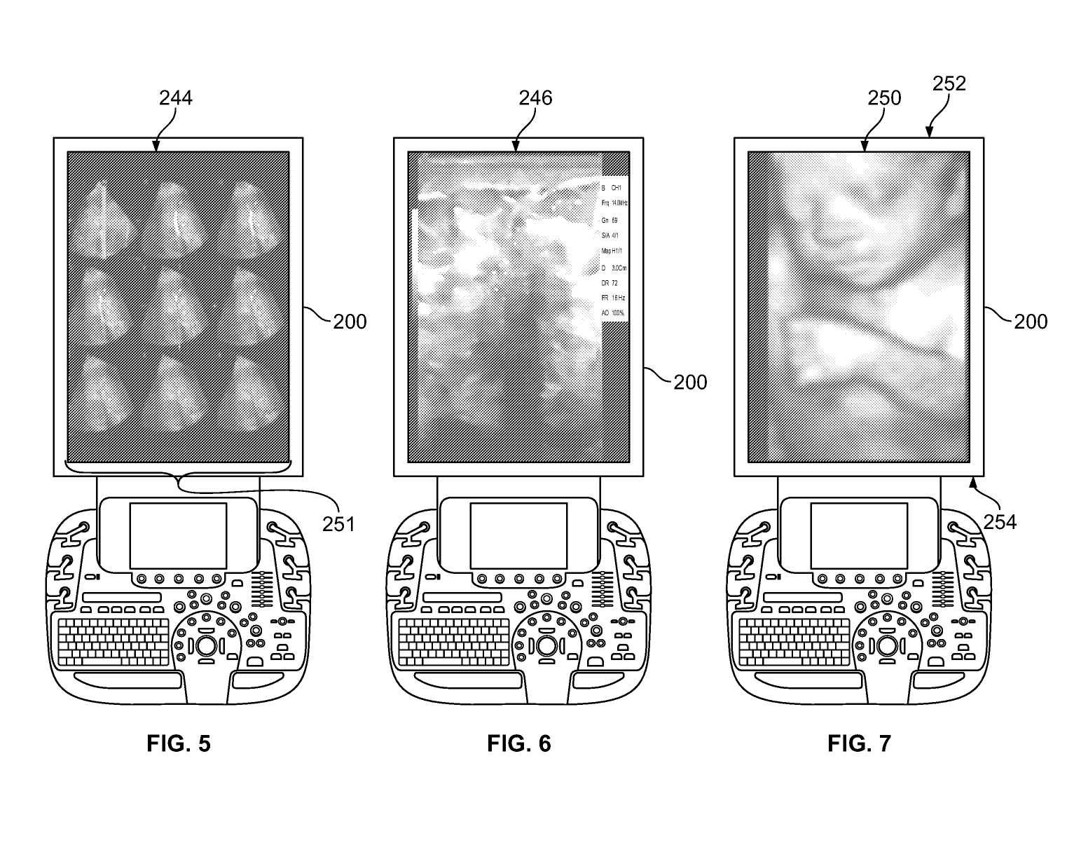

FIG. 5 illustrates a viewing configuration that may be used when the wide-screen display 200 is rotated to portrait orientation. For example, when the wide-screen display 200 is rotated from the landscape orientation of FIG. 4 to the portrait orientation of FIG. 5, the nine images 241 of FIG. 4 may be displayed across all or most of display area 251 in a different viewing configuration having a larger format of nine images 244. Therefore, when the processor module 116 detects, such as by a signal from or change in state of the position sensor 120, that the wide-screen display 200 has been rotated, the processor module 116 (of FIG. 1) may automatically update the viewing configuration of the wide-screen display 200 to display the nine images 244 based on operator preference and/or the protocol being used.

FIG. 6 illustrates another viewing configuration that may be used when the wide-screen display 200 is in portrait orientation. A larger format image 246 of the image 242 of FIG. 4 may be displayed. In this embodiment, when the wide-screen display 200 is rotated from the landscape orientation of FIG. 4 to the portrait orientation of FIG. 6, based on a viewing configuration the processor module 116 may automatically update the wide-screen display 200 to display the larger format image 246 rather than the nine images 244 as in FIG. 5. The workflow and ease of use may be enhanced for the operator because in the portrait orientation greater imaging depth may be displayed or the data may be displayed in a larger scale compared to the landscape orientation.

In yet another embodiment, the operator may choose a primary viewing configuration or desired display when the wide-screen display 200 is rotated between the landscape and portrait orientations. For example, when the wide-screen display 200 is rotated to the portrait orientation, the larger format image 246 may be displayed first and then the operator may select a button, which may be on the user interface 140 or may be a GUI button (not shown) on the wide-screen display 200, to toggle between the larger format image 246 and the display configuration showing the nine images 244. When the operator rotates the wide-screen display 200 to the landscape orientation of FIG. 4, the processor module 116 receives a signal from, or detects a change in the state of, the position sensor 120 and updates the wide-screen display 200 to display the image 242 and the nine images 241 simultaneously.

FIG. 7 illustrates another example of a viewing configuration for displaying image data when the wide-screen display 200 is in portrait orientation. A single image 250 is displayed. In this example, the review of the image 250 may be more intuitive in the portrait orientation than in the landscape orientation. Another example is when scanning with the transducer 106 in a sweeping motion, such as along a patient's leg to view vessels there-within. The acquired data may be intuitively displayed on the wide-screen display 200 in a format that enables easier interpretation. For example, the image may be displayed from top 252 to bottom 254 of the wide-screen display 200 as it is collected. In some embodiments, the larger size of the wide-screen display 200 may enable the operator to display images that are actual or approximate in size to the actual tissue and anatomy being scanned (or display all of the object being scanned instead of just a portion of the object).

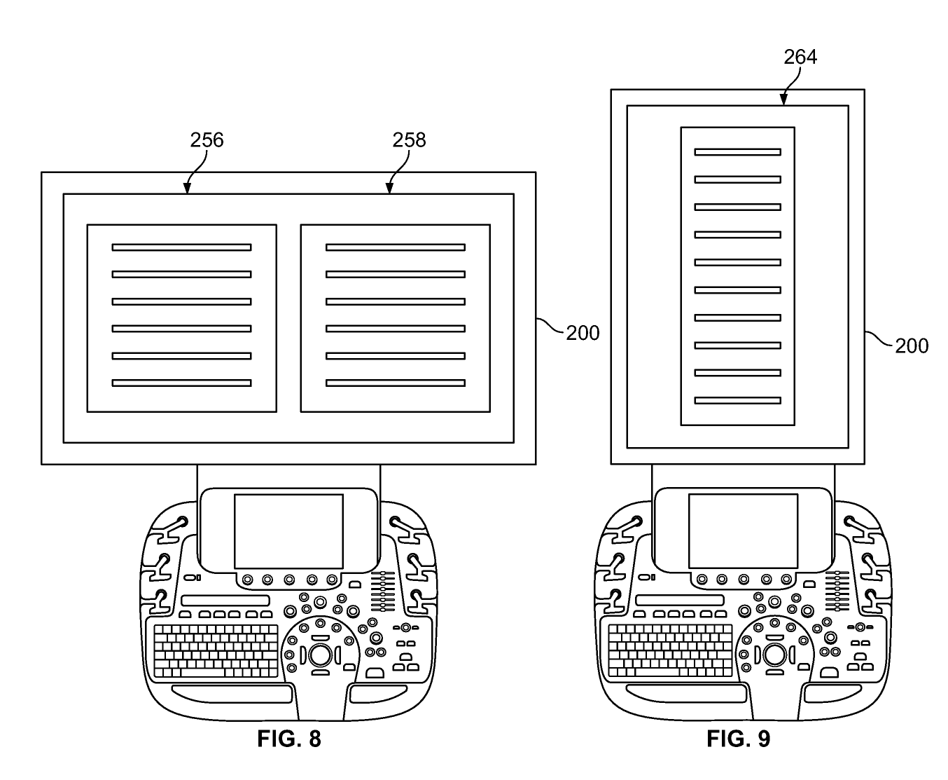

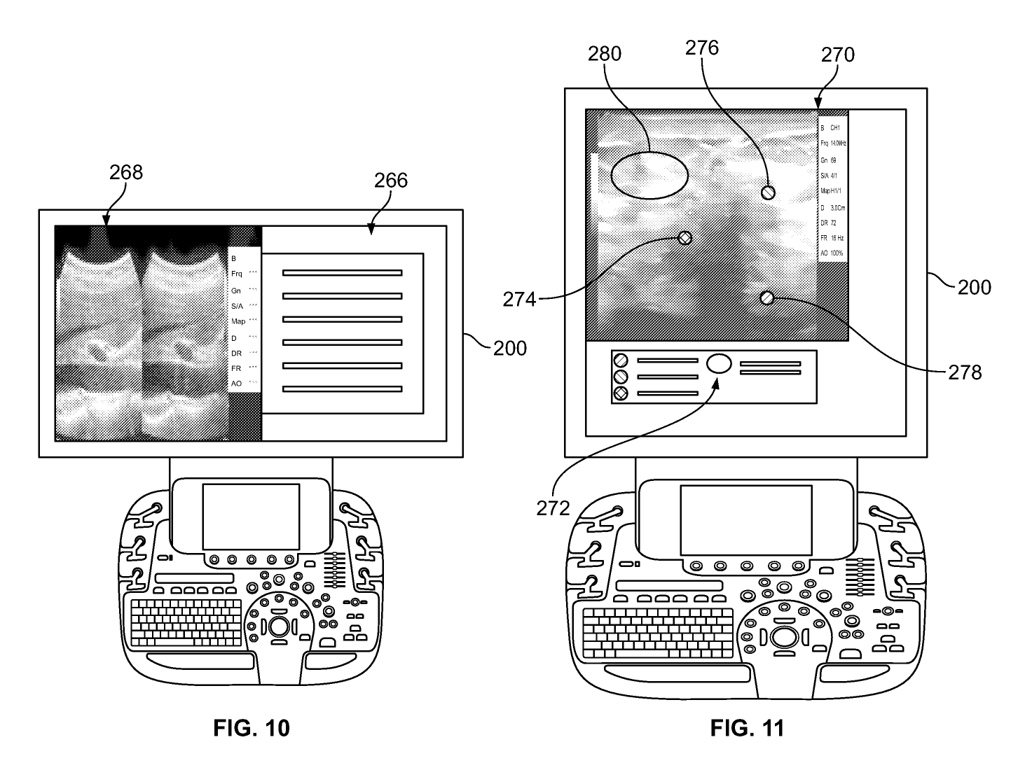

In another embodiment, the relatively larger size of the wide-screen display 200 allows more text associated with patient information to be displayed at the same time. Previously, the operator may have had to input data on multiple screen images, requiring the operator to page or move forward and backward within the input pages. FIG. 8 illustrates a viewing configuration of the wide-screen display 200 in landscape orientation. Rather than showing a single page of text, first and second pages 256 and 258 can be displayed simultaneously. Therefore, in landscape orientation the patient information may be displayed in a book format, such as displaying two or more “pages” side-by-side. FIG. 9 illustrates a viewing configuration of the wide-screen display 200 in portrait orientation, displaying the first and second pages 256 and 258 of the landscape orientation in a single list format 264. FIG. 10 illustrates yet another viewing configuration wherein the wide-screen display 200 is in landscape orientation, displaying a page of text 266 and one or more patient images 268 simultaneously.

FIG. 11 illustrates a viewing configuration that uses the wide-screen display 200 to display annotations associated with a patient image separately from the image. It should be understood that although a single image is shown, more than one image may be used. While in the portrait orientation as shown, image 270 may be displayed simultaneously with annotation section 272. Previously, operators select one or more points in the image and the associated annotation would be displayed near the point, potentially obscuring other data of interest within the image. In FIG. 11, the operator may select points on the image 270 that are shown with indicators 274, 276, 278 and 280. The indicators 274-280 are illustrated as open circles, but it should be understood that any indicator shape and/or color may be used to coordinate the position of the indicator on the image 270 to the corresponding indicator within the annotation section 272. The text associated with each of the indicators 274-280 is thus provided in an area separate from the image 270, improving the workflow and ease of use for the operator. Although the wide-screen display 200 is illustrated in the portrait orientation, it should be understood that the information may be displayed in the landscape orientation as well, such as in a viewing configuration that positions the image 270 and the annotation section 272 side-by-side within the display area.

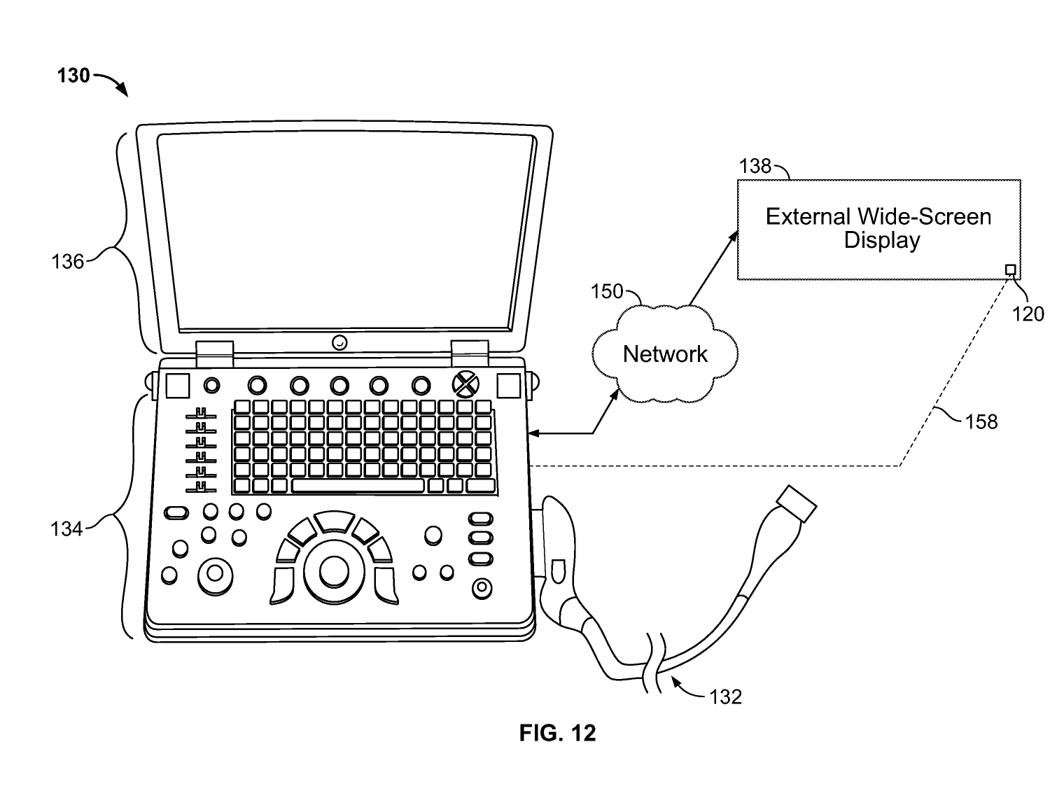

FIG. 12 illustrates a miniaturized ultrasound system 130 having a transducer 132 configured to acquire ultrasonic data. For example, the transducer 132 may have a 2D array of transducer elements 104 as discussed previously with respect to the transducer 106 of FIG. 1. A user interface 134 (that may also include an integrated display 136) is provided to receive commands from an operator. As used herein, “miniaturized” means that the ultrasound system 130 is a handheld or hand-carried device or is configured to be carried in a person's hand, pocket, briefcase-sized case, or backpack. For example, the ultrasound system 130 may be a hand-carried device having a size of a typical laptop computer, for instance, having dimensions of approximately 2.5 inches in depth, approximately 14 inches in width, and approximately 12 inches in height. The ultrasound system 130 may weigh about ten pounds, and thus is easily portable by the operator. It should be noted that the various embodiments may be implemented in connection with a miniaturized ultrasound system having different dimensions, weights, and power consumption.

The ultrasonic data and other associated diagnostic data and/or images acquired and/or accessed by the system 130 may be sent to an external wide-screen display 138 via a wired or wireless network 150 (or direct connection, for example, via a serial or parallel cable or USB port). The wide-screen display 138 may have the position sensor 120 and be, for example, free standing and rotatable as discussed in FIG. 1. The position sensor 120 may be integrated with the wide-screen display 138 or may be external and removable, allowing the operator to mount the position sensor 120 on different external wide-screen displays 138. The position sensor 120 may communicate via a link 158 that may be hard-wired to the system 130 or may communicate wirelessly with the system 130. Therefore, the system 130 may be configured to display images and data on the wide-screen display 138 in the viewing configurations and/or display formats as previously discussed. The wide-screen display 138 is rotatable between the portrait and landscape orientations and thus improves the imaging and workflow for the operator while allowing a high level of portability for the system 130 and flexibility in adding additional components, such as the wide-screen display 138, as needed.

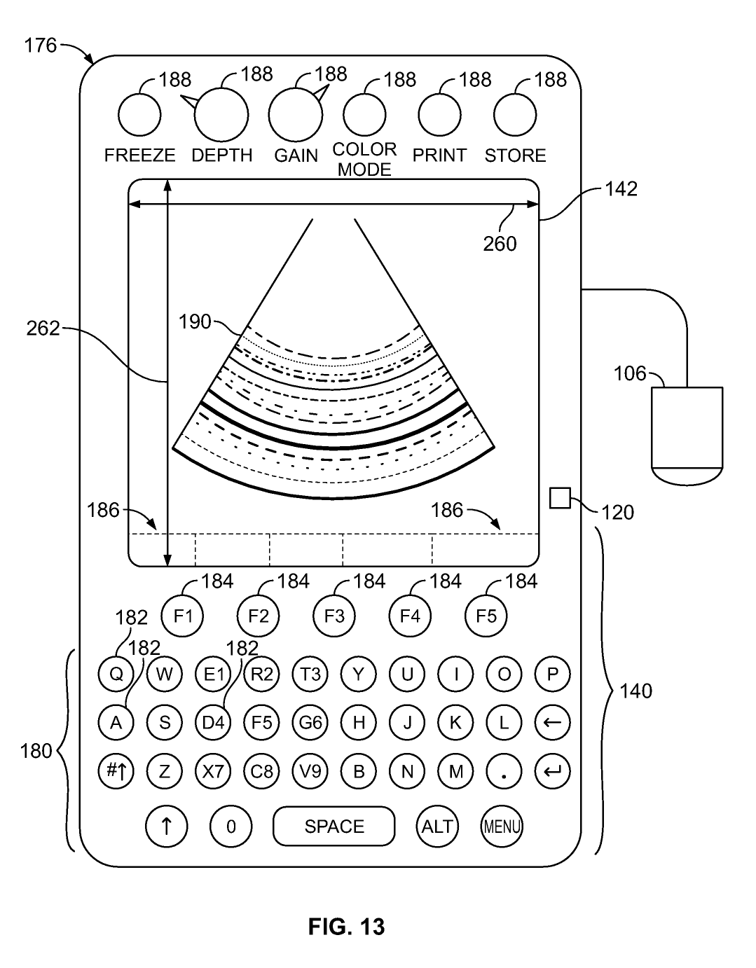

FIG. 13 illustrates a hand carried or pocket-sized ultrasound imaging system 176 wherein display 142 and user interface 140 form a single unit. By way of example, the pocket-sized ultrasound imaging system 176 may be a pocket-sized or hand-sized ultrasound system approximately 2 inches wide, approximately 4 inches in length, and approximately 0.5 inches in depth and weighs less than 3 ounces. The display 142 may be, for example, an LCD display (on which a medical image 190 may be displayed). In one embodiment, first and second dimensions 260 and 262 may be the same or approximately the same. In another embodiment, the first and second dimensions 260 and 262 may be different with respect to each other. A typewriter-like keyboard 180 of buttons 182 may optionally be included in the user interface 140.

The position sensor 120 may be provided within the system 176 to sense the orientation with which the operator is holding the system 176. In this example, the position sensor 120 may sense the orientation with respect to the ground. The position sensor 120 may sense that the operator has turned the system 176 from an approximately upright position to a tilted or rotated position and thus automatically changes the orientation to present the ultrasound information in a predetermined format. The predetermined format may be to simply rotate the image 190 by 90 degrees or may be to present a different image, group of images, touchscreen button(s) and the like based on the application. By automatically updating the display 142 based on the orientation, the workflow and operator viewing experience is improved.

Multi-function controls 184 may each be assigned functions in accordance with the mode of system operation. Therefore, each of the multi-function controls 184 may be configured to provide a plurality of different actions. Label display areas 186 associated with the multi-function controls 184 may be included on the display 142. The system 176 may also have additional keys and/or controls 188 for special purpose functions, which may include, but are not limited to “freeze,” “depth control,” “gain control,” “color-mode,” “print,” and “store.” In one embodiment, a multi-function control 184 or other input may be provided for toggling the automatic image rotation or change of viewing configuration on and off. For example, in some situations the operator may not want the image 190 to be rotated regardless of the orientation of the system 176.

A technical effect of at least one embodiment is the ability to rotate a wide-screen display to change the viewing experience of an operator of a medical diagnostic system. The system may display imaging and patient data in one viewing configuration while the wide-screen display is in a landscape orientation and in a different viewing configuration when the wide-screen display is in a portrait orientation. The operator may predefine viewing configurations based on the protocol being used or may choose a default viewing configuration. The larger display size of the wide-screen display allows more data (images and/or text) to be displayed simultaneously and/or more intuitively for the operator. Also, imaging data from different modalities may be displayed simultaneously such as for review and comparison.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A medical imaging system, comprising:
a detector for acquiring imaging data associated with a subject;
a display having first and second dimensions;
a position sensor for detecting an orientation of the display, the orientation being associated with the first and second dimensions; and
a processor module for determining the orientation of the display based on the position sensor, the processor module displaying information on the display in a first viewing configuration when the display is in a first orientation and in a second viewing configuration when the display is in a second orientation.

2. The system of claim 1, wherein the display is a wide-screen display and wherein the first and second dimensions are different with respect to each other.

3. The system of claim 1, wherein the medical system is one of a console ultrasound system, a hand-held ultrasound system, and a miniaturized ultrasound system.

4. The system of claim 1, wherein the position sensor is one of an electromechanical apparatus and a physical switch.

5. The system of claim 1, wherein the first and second viewing configurations are associated with at least one of a viewing and acquisition protocol.

6. The system of claim 1, wherein the first and second viewing configurations comprise at least one of different patient data, different images of a patient, and different locations of data and images on a display area of the display.

7. The system of claim 1, wherein the first and second viewing configurations are rotated by 90 degrees with respect to each other.

8. The system of claim 1, further comprising a memory for storing the first and second viewing configurations, the first and second viewing configurations being associated with at least one of a protocol and an operator.

9. The system of claim 1, wherein the medical system is one of a console ultrasound system, a hand-held ultrasound system, and a miniaturized ultrasound system, the display being further configured to display images acquired with an imaging modality that is different than ultrasound.

10. An ultrasound imaging system, comprising:
a transducer for acquiring ultrasound imaging data associated with a subject;
a wide-screen display having first and second dimensions that are different with respect to each other;
a position sensor for detecting an orientation of the wide-screen display; and
a processor module for determining the orientation of the display based on the position sensor, the processor module displaying information on the wide-screen display in a first viewing configuration when the wide-screen display is in a first orientation and in a second viewing configuration when the wide-screen display is in a second orientation.

11. The ultrasound imaging system of claim 10, wherein the wide-screen display is separable from the ultrasound imaging system.

12. The ultrasound imaging system of claim 10, wherein the first and second viewing configurations display the information on the wide-screen display in at least one of different display sizes and different font sizes with respect to each other.

13. The ultrasound imaging system of claim 10, wherein the first and second orientations are associated with landscape and portrait orientations, respectively, of the wide-screen display.

14. The ultrasound imaging system of claim 10, wherein the position sensor is one of an electromechanical apparatus and a physical switch.

15. A method for changing a viewing configuration based on a position of a display associated with a medical imaging system, comprising:
acquiring medical imaging data associated with a subject;
identifying a first position of a display, the first position being associated with one of a landscape orientation and a portrait orientation;
displaying information on the display based on the first position and a first viewing configuration;
identifying a second position of the display that is rotationally different with respect to the first position; and
displaying the information on the display based on the second position and a second viewing configuration that is different than the first viewing configuration.

16. The method of claim 15, further comprising defining the first and second viewing configurations based on at least one of a protocol and an operator.

17. The method of claim 15, wherein the first viewing configuration defines at least two display areas for displaying the information and the second viewing configuration defines one display area for displaying the information.

18. The method of claim 15, wherein the first viewing configuration displays the information based on at least two different imaging modalities and the second viewing configuration displays information from one imaging modality.

19. The method of claim 15, wherein the information being displayed based on the first position is rotated 90 degrees to form the information being displayed based on the second position.

20. The method of claim 15, wherein the display is one of integral with and externally interconnected with the medical imaging system, and wherein the medical imaging system is one of a console ultrasound system, a hand-held ultrasound system, a miniaturized ultrasound system, an X-ray system and a computed tomography system.
